(19) United States
(12) Patent Application Publication
(10) Pub. No.: US 2022/0042960 A1
(43) Pub. Date: Feb. 10, 2022

(54) METHOD FOR DETERMINING PROPERTIES OF FOODS

(71) Applicants: Christian Fleck, Heitersheim (DE); tsenso GmbH, Stuttgart (DE)

(72) Inventors: Matthias BRUNNER, Stuttgart (DE); Christian Fleck, Heitersheim (DE)

(21) Appl. No.: 17/393,620

(22) Filed: Aug. 4, 2021

(30) Foreign Application Priority Data
Aug. 5, 2020 (EP) 20189656.0

(51) Int. Cl. G01N 33/02; G06N 7/00; G06K 9/62; A23L 3/00

(52) U.S. Cl. CPC G01N 33/02; G06N 7/005; A23V 2002/00; G06K 9/6226; A23L 3/003; G06K 9/6297

(57) ABSTRACT

A method utilizing a digital twin instance in relation to food to query current/future properties thereof. A digital twin instance representing the food is generated from a digital twin template. The digital twin instance has assigned thereto, for a first target variable describing a food property, a mathematical model with a model parameter and an environmental parameter. The digital twin instance has a probability distribution for the model parameter of the first target variable. In the course of the handling of the food until it reaches a shop and/or at the shop, a measurement of the parameter is made, the values thereof being stored and assigned to the twin instance. The mathematical model of the first target variable, the probability distribution of the model parameter and the values of the environmental parameter are used to ascertain a probability distribution with respect to the target variable.

### CROSS-REFERENCE TO RELATED APPLICATION

This claims priority from European Application No. 20189656.0, filed Aug. 5, 2020, the disclosure of which is hereby incorporated by reference in its entirety.

### AREA OF APPLICATION AND PRIOR ART

The invention relates to a method for determining properties of foods.

Foods usually have only a limited shelf life. Their quality deteriorates from the moment they are produced, and what can be affected by this deterioration are both properties of secondary importance to health, such as changes in flavour, colour or consistency, and properties relevant to health with consumption, such as bacterial contamination and propagation and also formation of toxic substances.

To inform the consumer for how long a food is consumable without substantial loss of taste and quality and without a risk to health, many foods specify a best before date, which is usually applied to the food packaging during the production of the food. In the case of highly perishable foods with a particularly high risk to health when used too late, what is commonly specified instead of a best before date is a use by date, after which consumption should definitely not take place. Said risk primarily arises from so-called pathogens, which cause diseases when the concentration of said pathogens is excessively high owing to propagation in the food.

The moment of application of the particular date when producing or packaging the food does not make it possible to take into account here the subsequent handling of the food. This concerns both the handling of the food between the production or packaging facility and the shop, for example a supermarket, and the handling of the food following sale at the shop, i.e. for example when transporting the food from the supermarket to the home of the purchaser and when the food is stored by the purchaser, especially in the refrigerator of the purchaser.

DE 60217329 T2 discloses estimation of the quality of foods by capturing their temperature in the supply chain and ascertaining a current microbial count on the basis of the temperature profile and an initial microbial count. Warning messages are additionally generated depending on the temperature profile.

In practice, it is, however, difficult to calculate the current microbial count on the basis of an initial microbial count, since a specific initial microbial count can be established only with difficulty and since it is not possible to predict the behaviour of the pathogens at different temperatures to the extent that would be required to realize the proposals of DE 60217329 T2.

### OBJECT AND ACHIEVEMENT

It is an object of the invention to provide a method which allows to determine properties of foods.

To this end, what is provided by the method according to the invention is that, in relation to a food, a digital twin instance as a representation of the food is generated from a digital twin template.

At least the following items of information are assigned to said digital twin instance:

For at least one first target variable serving to describe a property of the food, the twin instance has a mathematical model which, for its part, has at least one model parameter and at least one environmental parameter.

Furthermore, the digital twin instance has at least one probability distribution with respect to the at least one model parameter of the mathematical model of the first target variable.

In the context of this invention, a probability distribution is understood to mean a function which assigns to each value of the associated model parameter a probability of its occurrence, with different probabilities being given for at least two possible values of the associated model parameter.

In the course of the handling of the food until it reaches a shop and/or at the shop, at least one measurement of the at least one environmental parameter is made, the values of the environmental parameter ascertained here being stored such that they are assigned to the twin instance. In this way, the twin instance is supplemented with data about the environment to which the actual food has been exposed.

To determine properties of the food, the mathematical model of the target variable, the probability distribution of the at least one model parameter and the ascertained values of the at least one environmental parameter are used to ascertain for the current point in time or a future point in time a probability distribution with respect to the at least one target variable.

What is thus provided by the method according to the invention is that the digital twin instance, which is a structure of digital data that has been assigned to a food or batch thereof, provides a tool which can provide practically usable information at any time as to whether and, optionally, for how long the food has a certain quality with respect to a target variable, usually a good quality, i.e. is not of concern for health and/or not objectionable in terms of aesthetics or taste.

The method according to the invention does specifically not attempt to make an absolute statement about quality.

An absolute statement, such as, for example, “The Listeria concentration in the food is X” or “The Listeria concentration in the food is above Y”, cannot in practice be reliably made for all individual foods of a batch, especially not when such a statement simply comes about on a mathematical consideration of the temporal change of target variables on the basis of model parameters which concern simple statistical mean values.

The method according to the invention, however, functions with respect to at least one model parameter of the mathematical model of the at least one target variable with the stated probability distribution and consequently also calculates the target variable as a probability distribution.

Besides the fact that the calculation of the target variable as a probability distribution better reflects the truth about the food in question than a calculation of an absolute value, which is generally likely to be incorrect as regards content, the probability distribution of the target variable allows adapted statements in relation to more specific questions concerning quality.

For example, it is possible to derive from the target variable not only that the food is, on statistical average, of good or outstanding quality, but also with what probability this is not the case and the food is possibly no longer edible. Such a circumstance also allows differentiated answers to the question of the usability of foods. For instance, owing to the stronger constitution of adults compared to toddlers, a food which is only, on statistical average, of good quality may be safely consumed by adults, whereas toddlers should not consume the food owing to the nevertheless excessively high probability of poorer quality.

The fact that the method according to the invention calculates the target variable as a probability distribution does not stand in the way of an absolute statement. Especially for information processing for end customers, for example on the customer's mobile phone after scanning of a QR code on the food, a probability distribution is usually not very appropriate, since a preferably clear statement is desired here. Here, it would therefore, for example, be preferable to specify, in relation to the target variable in question, the quality which is at least present for the best 99.99% of foods according to the probability distribution. In practice, it is generally also not helpful for the end customer to obtain a multiplicity of individual target variables; instead, they should result in a common rating such as, for example, “Until July 15 perfect in terms of aesthetics and taste when stored at 6° C., until July 30 safe as regards health for adults when stored at 6° C.”.

Preferably, for the purpose of outputting a more easily understandable output proceeding from the ascertained probability distribution of the target variable, use is made of the probability distribution with respect to the at least one target variable to ascertain, by adding up the area under the probability distribution, with what cumulated probability the target variable of the food is below or above a specified threshold for the target variable. Alternatively, use can be made of the probability distribution with respect to the at least one target variable to ascertain, by adding up the area under the probability distribution, what value of the target variable is statistically fallen short of or exceeded in the case of a specified proportion of the food.

Preferably, the digital twin instance comprises a mathematical model and at least one model parameter, which is assigned to the mathematical model and which is present in the form of a probability distribution, for at least one microbiological target variable. Microbiological target variables include in particular: concentration with respect to any pathogen, concentration of Listeria, concentration of Lactobacillales, concentration of Cronobacter, concentration of Bacillus cereus, concentration of Campylobacter, concentration of Salmonella, concentration of Shigella, concentration of Staphylococcus aureus, concentration of Pseudomonas spp., concentration of mould fungus and concentration of Aspergillus spp.

These are target variables, the evaluation of which is relevant to assessing whether the food can still be consumed safely as regards health.

Alternatively or additionally, the twin instance can, however, also comprise at least one model together with at least one model parameter in the form of a probability distribution that relates to a target variable of a biochemical nature, especially the degree of browning and/or the degree of ripeness, the acid content and/or the sugar content or the concentration of certain vitamins or oxidized fats. These are target variables relating to properties of secondary importance to health.

Likewise conceivable target variables of secondary importance to health that, however, are still relevant to the quality of the food are physical target variables such as, for example, colour, texture, water content, compressive strength and dry matter, and also more subjective or aggregated target variables such as taste or freshness.

The environmental parameters which are stored such that they are assigned to the twin instance comprise at least one environmental parameter specifying the temporal profile of a parameter of the environment to which the food has been exposed. This can be especially a temperature of the food and/or an ambient temperature in the room in which the food is stored. But also other environmental parameters such as, for example, the ambient air humidity in the room in which the food is stored may be relevant to the development of the target variables and are therefore captured and stored in the twin instance. A further environmental parameter which is sometimes of relevance in practice and which can optionally be stored such that it is assigned to the twin instance relates to the composition of the air surrounding the food during transport and storage. An example is the concentration of a trace gas (CO2, ethylene).

The digital twin instance is preferably generated at the earliest moment from which the food has reached its sale-ready state and from which monitoring of the at least one environmental parameter is possible.

For many food products, this means that the digital twin instance is generated at the moment of production, for example in a cutting plant in the case of meat products or during catching or at least while the catch is still being stored on the fishing boat in the case of fishery products.

In the case of those products in which the at least one environmental parameter, especially the storage temperature, can be captured starting from production, the digital twin instance is preferably formed as a kind of simple copy of the associated twin template, i.e. without the data in the digital twin instance already being adapted with respect to the specific food or its batch. Instead, it is only the definitions characteristic of the twin template and, in particular, the probability distributions of the model parameters that are based thereon that are inherited by the twin instance. The twin template is usually specific for the nature of the food and for the production plant. However, depending on the product, it may be additionally expedient if the twin template is also specific for an upstream farm, i.e. for example relates to pork cutlets from cutting plant A, which originate from pigs from pig breeding farm B. Measurement of target variables as early as in the production plant for the purpose of adaptation of the twin instance generated beforehand is usually not necessary in this procedure. Accordingly, the probability distributions of the model parameters of the digital twin instance of the food are preferably unchanged until at least at the moment at which the food leaves the production plant.

Tracking environmental parameters as early as from the moment at which the product reaches its sale-ready state is, however, practicable in any case. For instance, especially in the case of imported goods, for example fruit shipped from South America to Europe, there is, upon arrival in Europe, great improbability with respect to the target variables. Generating the digital twin instance solely on the basis of a digital twin template uniform for the product and its origin would usually not be useful in such a case. Instead, a target variable can be measured in the course of import, in the course of goods receipt and/or transfer of risk of a transport shipment of foods. The digital twin instance is then generated such that at least one probability distribution of at least one model parameter of the mathematical model of the target variable is stored in an adapted manner in the digital twin instance depending on the measurement result.

Preferably, at least one measurement of a target variable of the twin instance of the food is made in the course of the handling of the food until it reaches a shop and/or at the shop. This can be done in the manner descried above, for example directly during import. Alternatively or additionally, it can, however, also be done at a later time in the supply chain, for example in the form of an arrival measurement upon arrival at the shop.

Measurement of a target variable for adaptation of the twin instance can, however, also be done during the production of a food in order to approximate the digital twin instance even better to the reality of the represented food.

Besides direct measurement of a target variable of the twin instance, it is also possible to use indirect data relating to the description of the production details and relating to approximate determination of the target variable. For instance, instead of direct measurement of a microbial concentration on the product at the end of production, it might also be possible to use environmental hygiene, hygiene measurements on the machines and in the production spaces, if there are sufficient empirical values with respect to a correlation with the target variable. Furthermore, it is also possible to use a method from the field of digital image recognition in order to be able to deduce the target variable.

Depending on the result of the measurement, an update of the twin instance can be performed, especially an update of the probability distribution of at least one model parameter of the mathematical model of the target variable. In particular, an initial value of the target variable can be adapted on the basis of such a measurement. One method acknowledged for such a purpose is the Bayesian updating method, the use of which is also proposed for the present case.

If the probability distribution of a model parameter in the twin instance is adapted on the basis of a measurement made, it is advantageous if the corresponding previously valid probability distribution of the model parameter is left in the memory for the purpose of later transparency, so that it can be gathered from the twin instance at which moment probability distributions with respect to the target variable occurred taking into account which probability distribution of the model parameters.

Besides the check and optional update of the twin instance on the basis of a measurement, an update of the twin template can also be performed on the basis of the results of said measurement and a plurality of further measurements of foods, the twin instances of which were derived from the same twin template.

Such an update can be performed in a fully automatic manner. However, this is generally not preferred. Instead, it is considered expedient if the data ascertained by such measurements are viewed by a person skilled in the art and he takes his expert knowledge into account in assessing to what extent the measurement data support an adaptation of the mathematical model stored in the twin template and/or of the probability distribution of at least one model parameter.

Besides the update of the twin instance or optionally even the twin template, the measurement, especially if it yields a value of the target variable that is of concern for health and that is improbable based on the probability of the target variable as ascertained on the basis of the twin instance, can be used in order to bring about effects on the twin instances of other foods as well, especially other foods which come from the same batch as that of the food measured.

Thus, it is possible in particular to effect an adaptation of other foods with respect to the probability distribution of the model parameters. If the measured target variable reveals a relevant health risk, it may be expedient to even generate a warning message, especially a warning message which is assigned to twin instances of other foods, so that the relevant warning message is output when target variables of said twin instances of other foods are queried.

The measurement may also bring about generation of shelf life information taking into account the intended or alternative types of usage, from which information it is possible to gather for how long the food or other foods of the same batch is still suitable despite measurement values of concern for the particular type of usage.

Use of the digital twin instance for estimation of the values of the particular target variable for a specific food or batch thereof occurs especially prior to sale via individuals involved in the supply chain. For example, personnel at the shop can check the digital twins of incoming foods in order to be able to reject them if necessary or to be able to provide them with adapted end-customer information concerning shelf life.

In addition, the twin instances of different foods or different batches of the foods allow personnel along the supply chain to make decisions relating to priority in the handling of the foods. For example, a transport sequence in the event of limited transport capacities can be determined from the respective digital twins and from the probability distributions apparent therefrom with respect to the particular target variables. In addition, pricing at the shop can be done depending on the probability distribution of the target variable.

Furthermore, the goods of a transport chain can be quantified with respect to refrigeration or air humidity. In addition, the ascertained shelf life of the foods on the basis of the environmental variables actually measured can be related to the shelf life which would have been expected on the basis of the contractually agreed transport conditions. In addition, it is possible to make contractual agreements with transport companies which specify an at least achievable probability distribution of one or more target variables. This makes it simpler in practice to estimate difficult-to-avoid deviations from continuously ideal storage conditions correctly with respect to their effects on shelf life and to assess whether the handling by the transport company is nevertheless still according to contract and the food has the desired quality.

A further benefit may, however, also be that the ascertainment of the probability with respect to the at least one target variable using the twin instance can also be effected after triggering via a scanner of a customer or consumer at the shop or after purchase of the food. This allows the customer to make his purchase decision depending on the probability distribution of the target variable or, in the case of use of the foods in his refrigerator, to consider from when which foods with increased probability may no longer be consumed without health concerns.

The scanner can be especially a mobile phone which, via the camera or other sensors, allows the scanning of a food at the shop.

On the customer's scanner, what is preferably displayed is whether one or more target variables are within a safe range as regards health with a specified probability. Alternatively or additionally, it can be displayed on the customer's scanner with what probability one or more target variables are within a safe range as regards health.

Furthermore, it is preferred that the ascertainment of the probability with respect to the at least one target variable is done with inclusion of predicted or measured data relating to the at least one environmental parameter of the twin instance, wherein the customer or consumer provides for this purpose especially data which convey under what conditions the food was stored or will be stored and/or data which convey for how long and/or under what conditions the food was transported or will be transported until it reaches a cooling appliance of the customer or consumer. Appropriate data can also be already stored in the scanner, preset by the customer or provided in some other way.

If a warning message has been assigned to the twin instance, it is preferably output on the scanner. The customer can therefore be informed even after purchase of the food if measurements of other foods of the same batch have suggested an increased health hazard which warrants the assumption that other foods of the batch may be affected too.

The invention further relates to a computer program product or a computer system with a computer program product, wherein the computer program product comprises commands which, upon execution of the program by a computer, cause said computer to carry out the described method. A computer system for this purpose comprises various sub-components at various locations of a supply chain, as illustrated exemplarily on the basis of the following exemplary embodiments.

### BRIEF DESCRIPTION OF THE DRAWINGS

Further advantages and aspects of the invention are revealed by the claims and by the following description of preferred exemplary embodiments of the invention, which are elucidated below on the basis of the figures.

FIG. 1 shows the route of a food from the production facility into the refrigerator of a customer, and the information exchange taking place in the meantime with a server for generation and manipulation of a digital twin instance.

FIG. 2A to 2F show the digital twin instance generated in the course of the production of the food.

FIG. 3A to 3C show the probability distributions of model parameters of the digital twin instance.

FIG. 3D shows two exemplary temperature profiles, to which the food represented by the twin instance could be subjected.

FIG. 3E shows how different probability distributions arise after 240 hours with respect to the microbial load on the food depending on the different temperature profiles according to FIG. 3D.

FIG. 3F shows how it is possible to derive from the probability distributions statements concerning the suspected microbial load with different probabilities.

### DETAILED DESCRIPTION OF THE EXEMPLARY EMBODIMENTS

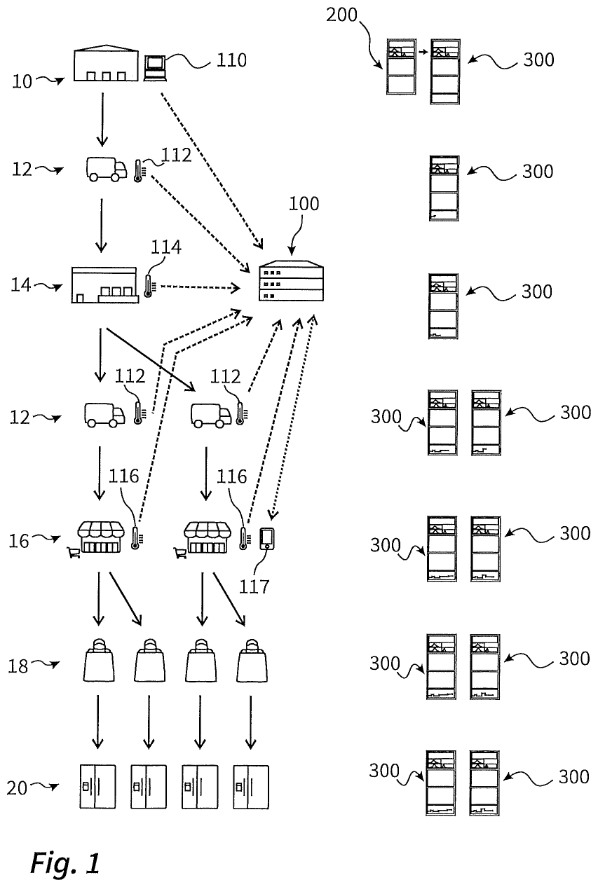

FIG. 1 shows the basic route of a food. Pork cutlets are produced in a production facility 10, for example a cutting plant, and from there, they are brought by means of, for example, trucks 12 with or without temporary storage in a refrigerated warehouse 14 to shops 16. Here, the product is purchased by customers, who bring the product in bags 18 to their respective homes, where the product is initially stored in refrigerators 20 until it is prepared or consumed.

To be capable at every point in time of assessing properties of the food, the food is accompanied on the route outlined in FIG. 1 by initially a digital representation 300, which is so to speak a digital twin of the food. Said digital twin, referred to hereinafter as digital twin instance 300, reflects how at least one target variable, preferably a plurality of target variables, changes over the lifetime of the food represented thereby.

A target variable is understood to mean a variable which is relevant to the quality of the food and especially the suitability of the food for consumption. Here, the variable can be especially variables relevant to health, such as, for example, a bacterial load. However, the variable can also be a variable of secondary importance to health, such as, for example, consistency or flavour. The units of target variables can be clearly defined units such as, for example, the number of colony-forming units of a certain microbial species per unit of weight of the food. However, arbitrarily chosen point units are also possible, for example a value within an interval between 0 and 100 that reflects the quality of taste, the highest quality of taste being represented by 100 points.

Said digital representation, the digital twin instance, is administered especially on a central server 100, to which the various systems yet to be described below have access, especially via the Internet, in order to generate the digital twin instance 300 or in order to retrieve or supplement data of said digital twin instance 300. The central server 100 is, in the usual fashion of today, preferably not a specific computer, but usually a server instance or a virtual server which operates in a computer centre on a multiplicity of interacting computers. Such an infrastructure is also commonly referred to as the cloud. It allows configuration of the system in an easily scalable manner. For simplification of language, a central server is, however, referred to hereinafter when what is meant is this central data administration.

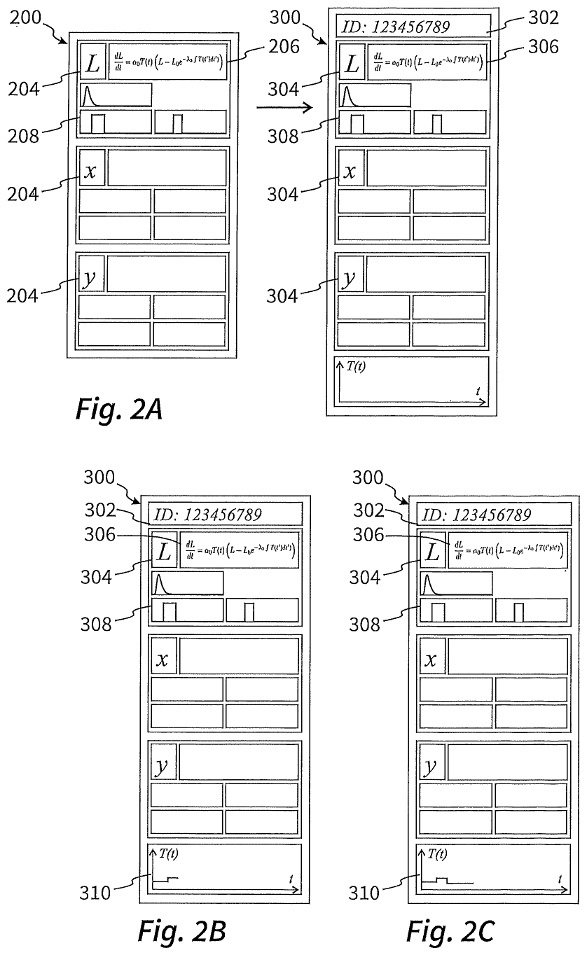

There is a template 200 for said digital twin instances, which was already generated beforehand independently of a specific food. Such a digital twin template 200 is preferably refers to a certain food type and a certain production facility. The digital twin template 200, which is depicted in FIG. 2A on the left-hand side, can be modelled prior to the production of a food on the basis of expert knowledge and experience thereof and on the basis of historical measurement data of the target variable and can, if necessary, be revised. However, it can also be derived automatically from past data and/or be automatically updated on the basis of newer data.

The digital twin template 200 primarily comprises two parts: firstly, at least one mathematical model 206 for calculation of at least one target variable 204, said mathematical model 206 comprising at least one model parameter and at least one environmental parameter, and secondly, a probability distribution 208 for the at least one model parameter.

Usually, one digital twin template 200 is provided for the estimation of multiple target variables 204. In this case, it has a distinct mathematical model 206 for each target variable 204 and at least one model parameter with a probability distribution 208 for each mathematical model.

However, for the sake of simplified illustration, what is considered in the present case is a digital twin template 200, for which there is a description with respect to the tracking of one target variable 204. The digital twin template 200 considered here is specific for a certain food, exemplified by pork cutlets in the present case, and a certain production facility, a fictional cutting plant A in the present case, characterized by the reference number 10 in FIG. 1. In principle, the twin templates 200 could, however, be provided in a more differentiated manner. For example, different twin templates 200 can be provided for different fattening farms.

The target variable 204 considered here, for the ascertainment of which the digital twin template 200 is inter alia provided, is the Listeria concentration L. Listeria are bacteria which occur ubiquitously in nature and feed on dead organic material. Therefore, Listeria can, even after the slaughter of animals, be found on the foods obtained therefrom, on pork cutlets in the present case. The unit used for such bacteria is the number of colony-forming units per gram of food (CFU/g).

The mathematical model used in the twin template 200 considered here is as follows:

$$\frac{d L}{d t} = {α}_{0} T ( t ) ( L - {L}_{0} {e}^{- {λ}_{0} {\int }_{{t}_{0}}^{t} T ( {t}^{′} ) {dt}^{′} )} )$$

This is a differential equation which has, on the left-hand side of the equation, the first derivative of the Listeria concentration with respect to time.

As can be seen from the model, it has furthermore three model parameters, namely the model parameters α₀, L₀ and λ₀. At least one of these model parameters, preferably all three model parameters, is not present in the form of specific values, but in the form of a probability distribution. The three model parameters represent the following items of information:

L₀ is the probability distribution of the initial microbial load with Listeria, which pork cutlets of the production facility 10 have, i.e. the cutting plant in the present case.

λ₀ is the probability distribution of the transition rate per unit of temperature, with which the Listeria on the cutlet on average transitions from an inactive lag phase into the growth phase.

α₀ is the probability distribution of the growth rate per unit of temperature, with which the Listeria propagate on the pork cutlet, if they are in the growth phase.

In practice, it is likely that use is made of more complex mathematical models with more model parameters. However, for the purpose of this description, the stated simplified mathematical model is sufficient.

With the production of a batch of pork cutlets in the production facility 10, a digital twin instance 300 for the entire batch is generated from the twin template 200, as illustrated in FIG. 2A. This means that, from a computer 110 in the product facility, which can, for example, also be a computer integrated in a barcode scanner, a request is transmitted to the central server 100 to generate a new twin instance data set in the memory. The central server 100 subsequently generates the twin instance 300 on the basis of the twin template 200, with acceptance of the mathematical model or mathematical models and the associated probability distributions of the associated model parameters of the twin template 200. What is possible here is copying of the mathematical model 206 or mathematical models 206 and the probability distributions of the model parameters into the twin instance 300. However, it is preferred that only referencing is done, i.e. the twin instance 300 contains a reference to the twin template 200.

Besides the mathematical model 306 or mathematical models 306 together with probability distribution 308 of the model parameters, the twin instance 300 additionally has a clear identification 302 in order to establish a clear connection to the food, initially primarily the batch in the present case. The clear identification 302 can be a batch number. However, other identifications are also conceivable. For example, one or more pallet identification numbers could be used as identification.

Besides the identification, further items of information can be stored in the twin instance that are essential items of information for the batch and are transmitted to the central server 100 especially from the computer 110 together with the request for generation of the digital twin instance, examples being the plant from which the pork originally came and/or the date of production in the cutting plant and/or the date of slaughter.

Furthermore, the twin instance 300 is provided with storage space which allows storage therein of environmental parameters and the temporal development thereof. In the present case, the only environmental parameter 310 required for the mathematical model for ascertainment of Listeria concentration is the parameter of temperature T and its change over time.

Starting with the production in the production facility 10, the particular given temperature data as relevant environmental parameter are transmitted to the central server 100 and stored in the twin instance 300. The temperature data can, for example, be captured in an automated manner in cold stores of the production facility 10, of the refrigerated warehouse 14 and of the shop 16 and be transmitted to the server 100 on the basis of the previously registered batch number of the foods stored therein. The same also applies to the transport to the shop in the trucks 12. In addition, it is naturally also possible in principle to make only assumptions in part along the route, especially on the basis of empirical values or, for example, values stored on cooling units in the cooling control system without the values being actually measured temperature values. The captured or known temperature data are likewise transmitted to the server 100 from data devices 112, 114, 116 with specification of the batch number or some other clear identifier and taken as supplementary data for the twin instance.

FIGS. 2B and 2C illustrate how the temperature data of the twin instance are supplemented by corresponding data of the transport in the truck 12 and of the refrigerated warehouse 14.

The pork cutlets of the batch are transported from the refrigerated warehouse 14 by truck 12 into different shops, FIG. 1 depicting exemplarily two such shops. Because there is thus a separation of the routes of different pork cutlets of the batch, a single twin instance is also no longer sufficient. The twin instance is therefore multiplied in line with the number of routes, this being illustrated in FIG. 1 and FIG. 2D by two digital twin instances 300 starting from the transport to the shops 16. The batch number used up to here for identification of the twin instance 300 is supplemented by a clear identifier with respect to the further route, for example by an identification of the shop 16. As explained above, the still united twin instance 300 might, however, also already contain multiple identifications, especially all the identifications of the pallets on which the batch is transported. In this case, after the twin instance has been duplicated, the two twin instances 300 can each contain as identification those pallet identifications belonging to pallets being further delivered to a particular shop.

Starting from the separation, the two twin instances 300 are each supplemented with temperature data on the server 100, said temperature data differing from one another owing to the different routes.

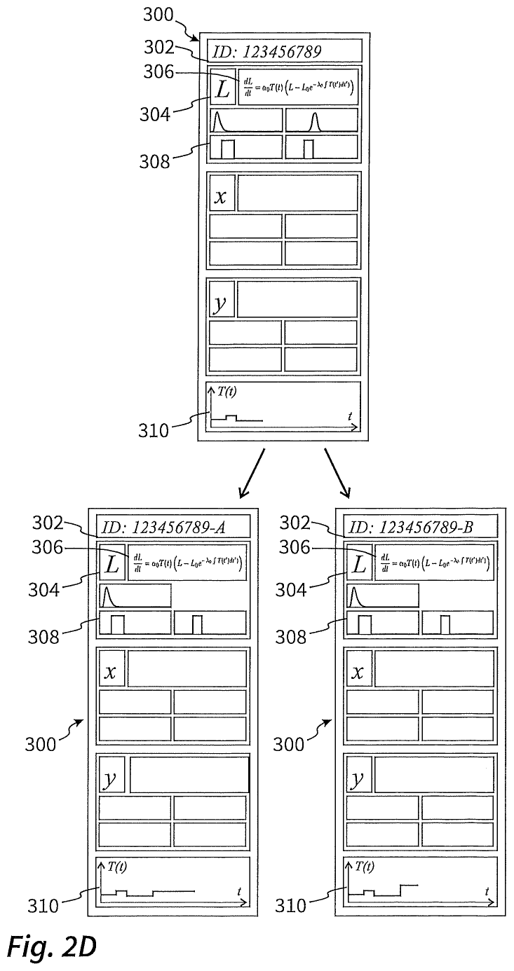

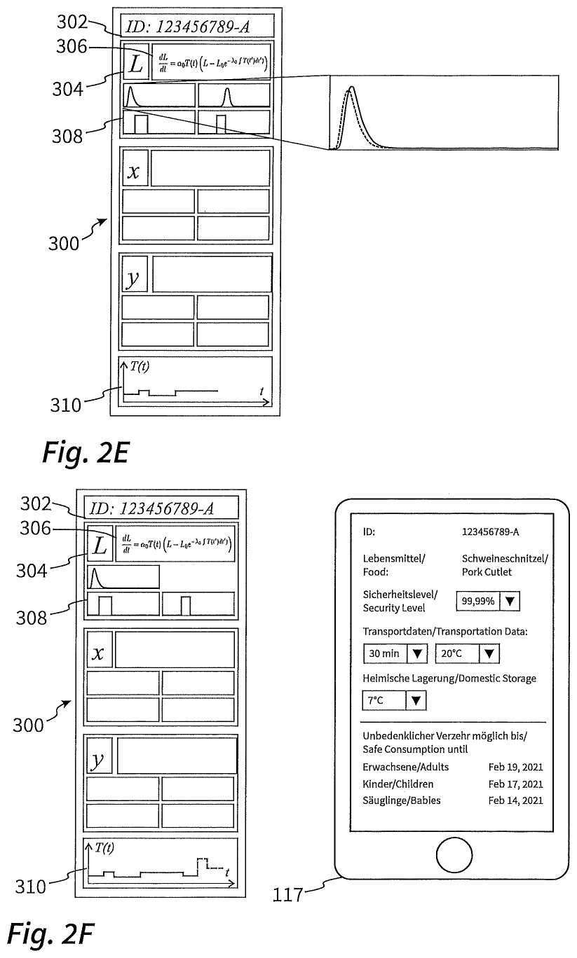

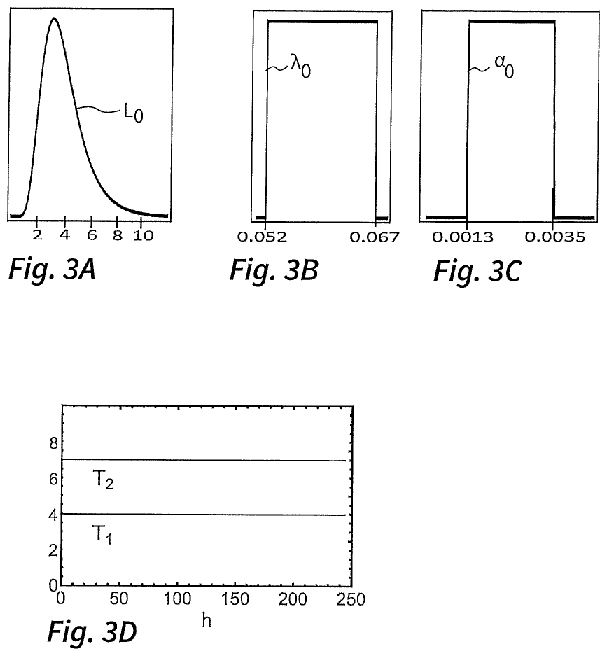

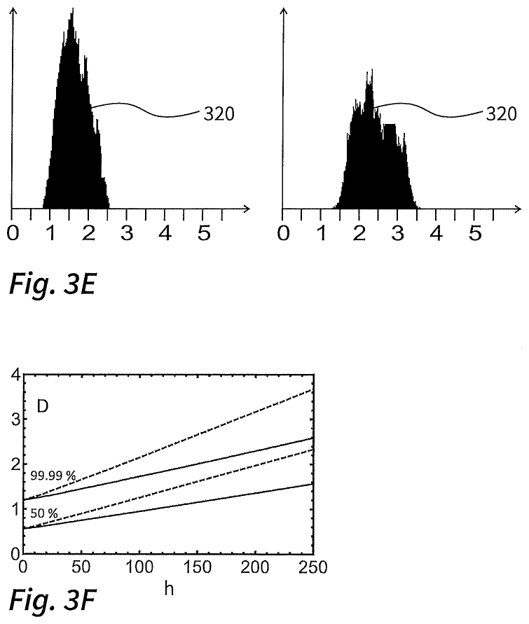

Proceeding from the temperature data appearing in each case in the various phases of the route of the food starting with the production facility, it is possible at any time to evaluate the food on the basis of the twin instance 300. Here, the target variables of the twin instance 300 are calculated, the result of this calculation being a probability distribution in each case. This will be illustrated in more detail below on the basis of FIG. 3A to 3E:

FIG. 3A to 3C show the distributions of the model parameters L₀, λ₀ and α₀, which, originating from the twin template 200, were used for creation of the twin model. As already mentioned, these probability distributions in the twin instance 300 are preferably, and especially preferably initially, only referenced and refer to the twin template 200. It can be seen that the probability distributions of the transition rate λ₀ and the growth rate α₀ are rectangular, i.e. that what is present in each case with the same probability is a transition value between 0.052 and 0.067 with respect to the transition rate λ₀ and from 0.0013 to 0.0035 with respect to the growth rate α₀. This rectangular shape of the probability parameters is probably not the actual probability distribution, but is due to the fact that only a confidence interval is known, within which the probability is greater than zero, though the exact distribution is not known and is not required either for estimation of Listeria by means of the mathematical model 306. The initial microbial load L₀ is, by contrast, more accurately known especially on the basis of earlier measurements and has, by contrast, a more non-uniform probability distribution with a peak at about 3 CFU/g, said probability distribution approximately resembling a logarithmic normal distribution. FIG. 3D shows a first possible temperature profile T₁ and a second possible temperature profile T₂ within the first 240 hours after production of the pork cutlet. The temperature profiles are greatly simplified in an exemplary manner, since what are considered are constant temperatures of T₁=4° C. and T₂=7° C. In practice, more complex temperature profiles would be included here.

Using the temperature profile T₁ or T₂ and the model parameters L₀, λ₀ and α₀ of the twin instance 300, it is possible to solve at any time the formula of the mathematical model 306. Since the model parameters or, according to the invention, at least one model parameter is present in the form of a probability distribution 308, the formula of the mathematical model 306 cannot be solved by single use. Instead, it is possible to use various values for the respective model parameters in separate calculation steps and to include them in the result taking into account their respective probability. In practice, this route is, however, not ingenious, since the same results can also be achieved with lower computing demand using suitable statistical methods, especially the Monte-Carlo method.

The Monte-Carlo method as well may occasionally not be sufficiently efficient, for example if many user requests must be processed in near real-time and this would be associated with a relatively great latency period between request and result. This is significant especially with respect to the end customer and the mobile app. In order to ensure an appropriately short response time, it is alternatively possible to resort to other mathematical methods such as spectral developments (generalized polynomial chaos expansion) or stochastic collocation.

The result is in turn a probability distribution, namely one of the probability distributions depicted in FIG. 3E. The probability distribution on the left is the probability distribution relating to the Listeria concentration L which appears over a period of 240 hours at a continuous temperature of T₁=4° C. The probability distribution on the right is that which appears over a period of 240 hours at a continuous temperature of T₂=7° C.

These probability distributions of FIG. 3E are, however, usually not the last step in the evaluation. On the contrary, such a probability distribution can be used in order to ascertain with what probability the pork cutlet has a load with the presently considered target variable of Listeria below a specified value, and so determinations can be made for different questions. For example, FIG. 3f shows Listeria concentration on the Y-axis and illustrates with the graphs, with what probability respectively 50% and respectively 99.99% are below the particular Listeria load over time. The dashed plots represent storage at a constant 7° C., whereas the solid plots represent storage at a constant 4° C.

For the described method, direct measurements of the at least one target variable, i.e. especially of Listeria concentration in the present case, on the batch in question are in principle not necessary. Solely on the basis of the digital twin and the mathematical model contained therein relating to Listeria concentration and also the stored model parameters, it is possible when the history of the environmental parameters, the temperature in the present case, is known to carry out an estimation relating to current Listeria concentration in the manner described.

Nevertheless, measurements of the target variable, of Listeria concentration in the present example, are expedient and usually take place along the distribution chain, especially during dispatch from the production facility 10 or the refrigerated warehouse 14 or during delivery at the refrigerated warehouse 14 or the shop 16. Depending on the nature of the measurement, it can then be used for updating of the twin instance in question and/or for indirect updating of the twin template.

Singular measurements on only one or a few pork cutlets of the batch should, in the event that the measured target variable, Listeria concentration in the present case, is within the range suggested by the digital twin 300, have no influence or no appreciable influence on the twin instance or the twin template.

However, if a more extensive series of measurements is carried out that largely rules out statistical deviations with respect to the actual value of the Listeria concentration L and if the measured Listeria concentration L is not within the plausible range based on the twin instance, as shown exemplarily by FIG. 3E, an adaptation of the twin instance 300 can be provided in this case. The twin instance is appropriately updated such that future calculations of Listeria concentration include the result of the measurement. The update will preferably be a correction of the probability distribution of the initial microbial load L₀ with Listeria. Since the updated probability distribution of the initial microbial load Lois a model parameter which, after the adaptation, now no longer corresponds with the corresponding probability distribution of the underlying twin template, the twin instance will preferably no longer reference the amended probability distribution of the initial microbial load L₀, but directly contain it as twin instance-specific data.

For updating of the probability distribution of the initial microbial load L₀ that was previously based on the twin template such that said probability distribution subsequently also includes the values of the measurement, suitable statistical methods are available, for instance the Bayesian updating method in particular.

Updating of the probability distribution and hence replacement of the original probability distribution need not take place immediately. It is also conceivable that initially only the measurement results are stored in the twin instance 300 and the adaptation of the probability distribution is only effected at a later time, especially if the probability distribution of the Listeria concentration is retrieved on the basis of the twin instance 300.

This is illustrated exemplarily by FIG. 2E for a series of measurements that is made upon arrival of the food at the shop 16. The series of measurements leads to an average Listeria concentration L which is distinctly lower than what would have been expected on the basis of the twin instance 300.

From the known temperature data of the environmental parameter of the twin instance 300, it is possible to deduce, on the basis of the measured average Listeria concentration, that the initial Listeria concentration L₀ was probably lower than had been assumed on the basis of the twin template 200. The result of this is that the probability distribution for the Listeria concentration L₀ in the twin instance is adapted, as illustrated by the dashed line in FIG. 2E. The updated probability distribution is, as previously mentioned, ascertained by means of the Bayesian updating method.

A measurement of the kind described, which has effects on the probability distribution of the initial microbial load L₀, can also already be made immediately after the production of the food, the pork cutlet in the present case. In this case, what can be concomitantly sent when transmitting the request from the computer 110 to the central server 100 are relevant measurement data which are stored in the twin instance 300 or immediately serve for adaptation of the probability distributions, for example on the basis of the use of the Bayesian updating method. Besides the effect on the twin instance 300, a measurement, especially in the form of the stated extensive series of measurements, can also influence the twin template 200. The measured data relating to the Listeria concentration L and also the temperature history since the production of the pork cutlet, which history is known and stored in the twin instance 300, allow, together with a multiplicity of further measurements on other batches of the same food product from the same production facility, adaptation of the probability distributions 208 of the model parameters. However, this is preferably not done automatically, but with examination and adaptation by experts.

The foods of the original batch are offered in the shops 16. While the foods are located there in the cooled window display, what is possible at any time via the particular digital twin is a check as to for how long the target variables, such as especially Listeria concentration, are within the permitted range.

This is especially also advantageous in the event of occurrence of unplanned warming of the foods due to faults. For example, if a cooling unit fails for a period of time, a check can be made taking into account what influence this has on the current shelf life and the predicted shelf life. Thus, after failure of the cooling unit, it is possible to make the decision, if necessary, that the food can no longer be sold or must be provided with a new best before date or use by date before it can be offered again.

For the purpose of checking by the customer, the foods can be provided with an identifier upon arrival in the shop, especially with the batch number supplemented by the shop, which is also stored in the digital twin 300. The identifier can, for example, be affixed in the form of a barcode or an NFC tag. It is also expedient when the current best before date and/or use by date in the light of the temperature data is attached to the food in readable form only upon arrival in the shop.

On the basis of the identification, the customers can, if needed, scan the food in question using a program, especially on their mobile phone 117, and thus access data of the digital twin 300 or data derived therefrom. Besides the simple retrieval of data stored in the digital twin, for example the temperature data, the customers can especially also retrieve the target variables. For instance, the customer can ascertain especially the probability with which the Listeria concentration is within a non-critical range for children and adults. Furthermore, the customer can, however, also apply a stricter standard and obtain information as to with what probability the Listeria concentration is also within a non-critical range for toddlers. What could be provided by another form of possible data presentation for the customer is for which target group, for example adults, adolescents, children, toddlers or infants, and for how long the food is unproblematic as regards health with a probability bordering on certainty, for example with a probability of at least 99.99%.

Furthermore, it is also possible for the customer to obtain a prediction relating to the target variable, which prediction depends on predicted future data relating to the environmental parameters, i.e. primarily temperature in the present case. For example, it is possible that the customer retrieves via the program on his mobile phone 117 a prediction as to for how long the Listeria concentration on the food still remains in the non-critical range when he transports the food home and to the refrigerator 20 within 30 minutes at the current ambient temperature and the food is then stored in the refrigerator at a temperature of 7° C.

The calculation can be done either by the server 100 or by the mobile phones 117. Storage of temperature data in the digital twin on the basis of temperature data predicted for the future usually does not occur. FIG. 2F illustrates, on the right-hand side, a possible query form on the mobile phone 117 of a customer. As already described, the customer can specify here predicted data relating to the environmental parameter of storage temperature, which data can be used in the calculation of the future Listeria concentration L and optionally other target variables. The twin instance 300 on the left-hand side in FIG. 2F illustrates this consideration by the predicted temperature profile depicted as dashes.

When the customer purchases a food, the route thereof separates from the foods of the same batch and thus of the same twin instance 300 that remain at the shop 16. In principle, it is conceivable that this is in turn associated with duplication of the twin instance. However, in practice, this will usually be too complicated, and so all further retrievals of target variables from this moment are preferably carried out using the twin instance with the state upon arrival in the shop 16.

However, if instead the twin instance is further duplicated, then it might be possible for the customer, after the purchase of the food, to continue to add temperature data to the twin 300 using the server 100 during the storage of the food in the refrigerator 20, in order to continue to be able to assess the quality of the food using actually specifically measured environmental data. If the customer transmits to the central server 100 such environmental parameters, especially temperature data, measured for the past, they could thus, in principle, be stored in a derived digital twin for the customer-purchased product. However, in this case, what would rather be preferred would be that these measured data are stored on the mobile phone 117 itself and are transmitted to the central server 100 in a temporary and optionally repeated manner only for calculation of the current properties, so that said server can ascertain target variables using the twin instance 300 upon arrival at the shop. To this end, the central server 100 need not permanently store the data captured by the customer.

The query form depicted in FIG. 2F is a rather complex query form. What presents itself in practice is providing the end customer with a simpler display in order to be able to check a food at the shop or later. For example, it could be limited to a display of lights or to a simple scale having a freshness value between 0 and 10. For conversion of the ascertained target variable to a corresponding value, an evaluation function is usually used. How this is specifically formed should also depend on the nature of the target variable. Target variables which relate to the concentration of pathogenic bacteria should be reflected in a summarizing evaluation such that it is already sufficient when one of the target variables with relevant probability is within an unacceptable range, in order to signal to the customer that the food should no longer be consumed. Target variables relating to variables which are of secondary importance to health and are, in particular, more variables relating to quality of consumption could be handled differently. For example, an evaluation function could add up various such target variables, and so a rather negative target variable could be compensated for by a rather high target variable.

The use of evaluation functions providing easily comprehensible results on the basis of one or more target variables is not limited to exclusive use on the mobile phone 117. In addition, such evaluation functions can, for example, also be used in order to set the price of the particular food at the shop. In the described example, what was used was the twin template 200 which is specific for the product of pork cutlets and for the specific production facility 10. However, it is alternatively also possible that there are multiple alternative twin templates and/or one twin template with multiple alternative model-parameter sets in order to take into account further factors, such as, for example, the farm from which the pig originates and/or the abattoir in which the pig has been slaughtered and cut into pig halves.

Various twin templates which are specific for different food products and production facilities and, optionally, also for further factors relating to origin are preferably not handled on the server as twin templates that are completely separate from one another, but are instead sorted into a hierarchy. For example, there can be a general twin template for pork products that forms the basis of various twin templates of different pork products. Said twin templates for different pork products can then, in turn, form the basis of the twin templates which are specific for the production facility and which, for their part, are used in order to be utilized in the above-described manner for deriving the twin instances.

Such a hierarchy makes it possible, for example, to assign fundamental mathematical models of various target variables to higher hierarchy levels and to take them therefrom in a uniform manner into lower hierarchy levels, whereas probability distributions of the respective model parameters are assigned to the lower hierarchy levels.

1. A method for ascertaining properties of foods, having the following features:
a. a digital twin instance as a representation of the food is generated from a digital twin template, and
b. at least the following items of information are assigned to the digital twin instance:
for at least one first target variable serving to describe a property of the food, a mathematical model which has at least one model parameter and at least one environmental parameter, and
a probability distribution with respect to the at least one model parameter of the mathematical model of the first target variable, and
c. in the course of the handling of the food until it reaches a shop and/or at the shop, at least one measurement of the at least one environmental parameter is made, the values of the environmental parameter ascertained here being stored such that they are assigned to the twin instance, and
d. the mathematical model of the first target variable, the probability distribution of the at least one model parameter and the ascertained values of the at least one environmental parameter are used to ascertain for the current point in time or a future point in time a probability distribution with respect to the at least one target variable.

2. The method according to claim 1, having at least one of the following further features:
a. use is made of the probability distribution with respect to the at least one target variable to ascertain, by adding up the area under the probability distribution, with what cumulated probability the target variable of the food is below or above a specified threshold for the target variable, and/or
b. use is made of the probability distribution with respect to the at least one target variable to ascertain, by adding up the area under the probability distribution, what value of the target variable is statistically fallen short of or exceeded in the case of a specified proportion of the food.

3. The method according to claim 1, having at least one of the following features:
a. the mathematical model of the digital twin instance of at least one target variable is a mathematical model for ascertainment of one of the following microbiological target variables:
concentration with respect to any pathogen and/or
concentration of Listeria and/or
concentration of Lactobacillales and/or
concentration of Cronobacter and/or
concentration of Bacillus cereus and/or
concentration of Campylobacter and/or
concentration of Salmonella and/or
concentration of Shigella and/or
concentration of Staphylococcus aureus and/or
concentration of Pseudomonas spp. and/or
concentration of mould fungus and/or
concentration of Aspergillus spp., and/or
b. the mathematical model of the digital twin instance of at least one target variable is a mathematical model for ascertainment of one of the following biochemical target variables:
degree of browning and/or
degree of ripeness and/or
acid content and/or
sugar content and/or
concentration of vitamins and/or
concentration of oxidized fats, and/or
c. the mathematical model of the digital twin instance of at least one target variable is a mathematical model for ascertainment of one of the following physical target variables:
colour and/or
texture and/or
water content and/or
compressive strength and/or
dry matter, and/or
d. the mathematical model of the digital twin instance of at least one target variable is a mathematical model for ascertainment of one of the following subjective or aggregated target variables:
taste and/or
freshness and/or
quality.

4. The method according to claim 1, having the following feature:
a. the environmental parameters which are stored such that they are assigned to the twin instance comprise at least one of the following environmental parameters:
temperature of the food and/or ambient temperature in the room in which the food is stored, and/or
ambient air humidity in the room in which the food is stored, and/or
composition of air surrounding the product.

5. The method according to claim 1, having the following feature:
a. the digital twin instance as a representation of the food is generated in the course of production in one of the following production plants
in a cutting plant in the case of meat products, or
during catching in the case of fishery products.

6. The method according to claim 1, having the following feature:
a. the digital twin instance as a representation of the food is generated in the course of goods receipt and/or transfer of risk of a transport shipment of foods.

7. The method according to claim 1, having the following features:
a. at least one measurement of a target variable of the twin instance of the food is made in the course of the handling of the food before it reaches a shop and/or at the shop, and
b. depending on the result of said measurement, an update of the twin instance is performed, especially an update of the probability distribution of at least one model parameter of the mathematical model of the target variable.

8. The method according to claim 7, having the following feature:
a. depending on the result of the measurement, an update of the probability distribution of the model parameter is performed concerning an initial value of the target variable during production of the food.

9. The method according to claim 7, having the following feature:
a. in the case of update of the at least one model parameter of the mathematical model of the target variable, the previously valid probability distribution of the model parameter is left in a memory of the twin instance for the purpose of later traceability.

10. The method according to claim 1, having the following features:
a. at least one measurement of a target variable of the twin instance of the food is made in the course of the handling of the food until it reaches a shop and/or at the shop, and
b. an update of the twin template is performed on the basis of the results of said measurement and a plurality of further measurements of foods, the twin instances of which were derived from the same twin template.

11. The method according to claim 1, having the following features:
a. at least one measurement of a target variable of the twin instance of the food is made in the course of the handling of the food until it reaches a shop and/or at the shop, and
b. if the measurement yields a value of the target variable that is of concern for health and that is improbable based on the probability of the target variable as ascertained on the basis of the twin instance, at least one of the following measures is taken, optionally in a dependent manner and/or differentiated manner according to the severity of the health concerns:
adaptation of other twin instances of other foods, especially other foods which come from the same batch as that of the food measured, and/or
generation of a warning message, especially a warning message which is assigned to twin instances of other foods, especially other foods which come from the same batch as that of the food measured.

12. The method according to claim 1, having the following feature:
a. the ascertainment of the probability with respect to the at least one target variable using the twin instance is effected after triggering via a scanner of a customer or consumer at the shop or after purchase of the food.

13. A computer program product or computer system having the following feature:
the computer program product comprises commands or the computer system comprises a computer program product with commands which, upon execution of the program by a computer, cause said computer to carry out the method according to claim 1.

14. The method according to claim 5, wherein the model parameters of the digital twin instance of the food are unchanged with respect to the digital twin template until at least at the moment at which the food leaves the production plant.

15. The method according to the claim 6, wherein in the course of the goods receipt or the transfer of risk of the food, at least one measurement of a target variable is made on at least one individual food and the digital twin instance is generated such that at least one probability distribution of at least one model parameter of the mathematical model of the target variable is stored in the digital twin instance depending on the measurement result.

16. The method according to claim 12, wherein the scanner is a mobile phone, and/or on the customer's scanner, what is displayed is whether one or more target variables are within a safe range as regards health with a specified probability, and/or on the customer's scanner, what is displayed is with what probability one or more target variables are within a safe range as regards health, and/or the ascertainment of the probability with respect to the at least one target variable is done with inclusion of predicted or measured data relating to the at least one environmental parameter of the twin instance, wherein the customer or consumer provides for this purpose especially data which convey under what conditions the food was stored or will be stored and/or data which convey for how long and/or under what conditions the food was transported or will be transported until it reaches a cooling appliance of the customer or consumer, and/or if a warning message has been assigned to the twin instance, it is output on the scanner.
